(19) United States
(12) Patent Application Publication
(10) Pub. No.: US 2013/0018221 A1
(43) Pub. Date: Jan. 17, 2013

(54) PENIS STRETCHING SYSTEMS

(71) Applicant: Justin Ball, Rahway, NJ (US)

(72) Inventor: Justin Ball, Rahway, NJ (US)

(21) Appl. No.: 13/352,838

(22) Filed: Jan. 18, 2012

Related U.S. Application Data
(60) Provisional application No. 61/433,578, filed on Jan. 18, 2011.

(51) Int. Cl. A61F 5/00; A61H 1/00

(52) U.S. Cl. 600/38; 601/46

(57) ABSTRACT

A penis stretching system designed to provide for safe, incremental stretching of a penis to increase a girth and length of the penis. The incremental stretching may be provided as Eukaryotic cells of the penis are torn and repaired. The penis stretching system comprises: a penis stretcher assembly including; a cylindrically-shaped frame; an adjustable securing strap; and at least one vibrator. The cylindrically-shaped frame includes a proximate end comprising a base engager; a distal end comprising a shaft engager; and telescoping legs. The vibrator provides vibration to provide comfort (and stimulation when needed) for a user-wearer. The penis stretcher assembly comprises the cylindrically-shaped frame, the adjustable securing strap and the vibrator in combination.

### CROSS-REFERENCE TO RELATED APPLICATION

The present application is related to and claims priority from prior provisional application Ser. No. 61/433,578, filed Jan. 18, 2011 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. FIELD OF THE INVENTION

The present invention relates generally to the field of penis devices and more specifically relates to a lengthening device which may be continuously worn by a male-user which may serve to enlarge the male-user's penis.

### 2. DESCRIPTION OF THE RELATED ART

Sex is a healthy and natural part of life. A physical expression of love, commitment, or simple passion, a healthy sex life may be invigorating and meaningful. For most sexually active men and women, sex involves the conscious, positive expression of sexual energy in ways that enhance self-esteem, physical health, and an emotional relationship. Mutually beneficial and pleasurable, consensual sex is the cornerstone for most coupled relationships. Because sex can be so physically and emotionally gratifying, a healthy sex life is a key component of most coupled partnerships and a natural part of life for many single people, as well. Medications may be used or surgery performed; however these options may prove dangerous to the individual's health and well-being.

Perhaps one of the most prevalent reasons many men feel sexually inadequate has to do with the size of their penis. Although often difficult to discuss, for many men, penis size contributes a great deal to their level of confidence and self-esteem and having a “small” penis can make a man feel inadequate and ashamed. Even men with adequate or larger penises may experience erectile dysfunction. Commonly referred to as ‘ED’, erectile dysfunction may result from factors ranging from psychological problems such as simple stress and generalized anxiety disorder to physical reasons stemming from prostate or bladder cancer surgery and aging. While these problems may be devastating for the man, they may also prove frustrating for their partners, who may end up feeling sexually unfulfilled. Therefore, a need exists for a male penis enlarger which my serve to lengthen and strengthen the male penis in a safe manner.

Various attempts have been made to solve the above-mentioned problems such as those found in U.S. Pat. Nos. 5,344,396 to Clark; 5,599,275 to France; 5,836,864 to Clark; 5,951,460 to Vollrath; and 7,566,299 to Montgomery. This prior art is representative of penis enlargement devices. None of the above inventions and patents, taken either singly or in combination, is seen to describe the invention as claimed.

Ideally, a penis stretching system should be safe and user-friendly and, yet would operate reliably and be manufactured at a modest expense. Thus, a need exists for a reliable penis stretching system to lengthen the penis of a user-wearer and to avoid the above-mentioned problems.

### BRIEF SUMMARY OF THE INVENTION

In view of the foregoing disadvantages inherent in the known penis accessory device art, the present invention provides a novel penis stretching system. The general purpose of the present invention, which will be described subsequently in greater detail is to provide a safe and comfortable penis stretching system.

A penis stretching system is disclosed herein, in a preferred embodiment comprising a penis stretcher assembly including a cylindrically-shaped frame; an adjustable securing strap; and two vibrators. The penis stretching system provides for safe, incremental stretching of the penis to increase a girth and length of the penis. Further, the penis stretcher assembly can be comfortably worn while a user-wearer is in a standing position and a non-standing position throughout an extended time duration.

The cylindrically-shaped frame includes a proximate end comprising a base engager; a distal end comprising a shaft engager; and telescoping legs. The penis stretcher assembly comprises the cylindrically-shaped frame, the adjustable securing strap and the vibrators in combination. The proximate end comprises a first half and a second half suitable as the base engager such that the proximate end is adjustable to fit about a penis base adjacent a scrotum. Further the proximate end is extendable and retractable in diameter via the first half and the second half of the base engager (movable in relation to each other) such that the proximate end is width adjustable to fit about the penis base adjacent the scrotum. The proximate end comprises a cammed upper surface in preferred embodiments.

The penis stretcher assembly further comprises removable studs, with the removable studs being able to be coupled to receiver inserts located in the cammed upper surface in the proximate end. The cammed upper surface in the proximate end additionally comprises guide tracks allowing the telescoping legs to move along the cammed upper surface. The distal end preferably comprises an arc comprising a right half-arc and a left half-arc. The right half-arc and the left half-arc are adjustable in relation to each other providing that the distal end is able to friction engage a penis shaft adjacent and under a knob.

The adjustable securing strap comprises rubber (or flexible, soft plastic, or the like) and the distal end in combination engage the penis shaft. The adjustable securing strap is held stationary by the left half-arc and is insertable through the right half-arc to create a contact-fit of the adjustable securing strap about the penis shaft. The adjustable securing strap is able to accommodate a penis, the penis being either circumcised or uncircumcised.

The proximate end and the distal end may be connected via the telescoping legs. The telescoping legs are able to provide extension and retraction of the penis stretcher assembly with relative ease. Additionally the telescoping legs are lockable via locking detents or other lockable fastening means. Locking detents may allow for locking at a plurality of positions in preferred embodiments. The telescoping legs comprise rigid rubber material, soft plastic or other suitable equivalent.

The vibrator(s) provide vibration to provide comfort for a user-wearer. The incremental stretching is provided as Eukaryotic cells of the penis are torn and repaired over an extended time period. The vibrators permit comfort-controlled incremental stretching of the penis and also promotes blood flow to decrease heal time. The proximate end and the distal end each may comprise vibrators comprising a first vibrator and a second vibrator, respectively. The first vibrator and the second vibrator(s) are independently activatable. The vibrators may each comprise activator buttons. The activator button(s) for the vibrator are adjustable to adjust a frequency of the vibration(s).

A method of using the penis stretcher assembly is also disclosed herein preferably comprising the steps of: inserting a penis into a penis stretcher assembly; securing an adjustable securing strap and a distal end to a shaft of the penis; securing a proximate end to a base of the penis; stretching the penis using telescoping legs throughout a time period; and vibrating the proximate end and the distal end while stretching the penis to increase blood flow to accelerate repair of cells torn during the stretching step.

The present invention holds significant improvements and serves as a penis stretching system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, a penis stretching system, constructed and operative according to the teachings of the present invention.

FIG. 1 shows a perspective view illustrating a penis stretching system in an in-use condition according to an embodiment of the present invention.

FIG. 2 is a perspective view illustrating a penis stretcher assembly of the penis stretching system in a retracted condition according to an embodiment of the present invention of FIG. 1.

FIG. 3 is a perspective view illustrating the penis stretcher assembly of the penis stretching system in an extended condition according to an embodiment of the present invention of FIG. 1.

FIG. 4 is a perspective view illustrating the penis stretcher assembly of the penis stretching system in the extended condition about a penis according to an embodiment of the present invention of FIGS. 1 and 3.

FIG. 5 is a flowchart illustrating a method of using the penis stretcher assembly of the penis stretching system according to an embodiment of the present invention of FIGS. 1-4.

The various embodiments of the present invention will hereinafter be described in conjunction with the appended drawings, wherein like designations denote like elements.

### DETAILED DESCRIPTION

As discussed above, embodiments of the present invention relate to a penis accessory/device and more particularly to a penis stretcher as used to improve the overall penis dimensioning and health.

Generally speaking, the perfect stretcher may comprise an assistive apparatus designed for use by the male population to help increase penis size and operative ability, without the use of medications such as Extenze and Viagra. The present invention may comprise a cyclindrical shape having a lower base unit which may serve to comfortably secure the unit underneath the scrotum. Telescopic rods may connect the base of the unit to the top of the unit which may serve to facilitate the actual stretching of the penis. The top portion of the Perfect Stretcher may comprise a recessed niche for placing the distal end of the penis. Further, an adjustable strap may serve to tie around this part of the penis, suitably securing the member to the unit. The adjustable strap may comfortably accommodate men who are circumcised or uncircumcised. The Perfect Stretcher is conceived to be worn on a regular basis. Over time, the length of the apparatus may be extended and retracted via the telescopic rods. In this manner, the muscles of the penis are able to stretch along with the present device disclosed herein.

The ‘Perfect Stretcher’ may comprise an extendable upper portion comprising two vertical openings for receiving an adjustable strap and a bottom portion comprising an extendable base having removable studs. Two sets of telescopic rods preferably connect the upper portion to the bottom portion. The upper portion and bottom portion may further comprise a pushable button located along one side. Upon pushing the button, the Perfect Stretcher may vibrate thus providing stimulation to the male penis. This may be utilized to assist the male user in erecting his penis to be optimally stretched.

When the Perfect Stretcher is in an extended position the telescopic rods may extend vertically thus stretching an inserted penis while in an in-use condition. When the telescopic rods are fully extended, the telescopic rods may be locked via an extendable locking shaft. The upper portion and bottom portion of the Perfect Stretcher are extendable thus enabling various penis sizes and girths to comfortably fit inside.

In use, the male-user inserts the penis through the upper portion so that the base of the penis comes into contact with the bottom of the studs located on an upper surface of the extendable base. The adjustable strap loops through the upper portion and may be secured around the head of the penis. The adjustable strap may be adjustably secured by the male-user such that the penis is securably fit inside the Perfect Stretcher. When the present invention is properly fitted to the male user's penis, the device may be extended and retracted via the telescopic rods. As such, the penis is continuously being stretched and pulled while in use. The continuous stretching and pulling of the penis consequently results in the strengthening of the penis muscle and the lengthening of the shaft. Preferably, the Perfect Stretcher should be worn and used by the male-user on a regular basis for optimal results.

The Perfect Stretcher preferably comprises removable studs providing stimulation to the male user. In use, the studs may work cooperatively with the vibration caused by pushing the button on either the upper or lower portion of the present invention to provide maximum stimulation with extra conditioning for the prostrate and maximum results. When the penis is fully erect, the Perfect Stretcher may serve to stretch the penis via intermittent pulling motions to strengthen and lengthen the penis.

The Perfect Stretcher may offer men a safe and simple means of increasing penis size and health; a therapeutic instrument which may be comfortably worn on a daily basis may accentuate the size of the penis, providing the male-user a sense of sexual confidence, while heightening their self-esteem and sense of virility. In addition, the Perfect Stretcher may prove extremely beneficial to men who suffer from erectile dysfunction (ED). Instilling confidence, the present invention may assist men who experience ED as a result of stress or anxiety. Moreover, use of this product may conceivably return men who suffer ED as a result of physical maladies to an active, happy sex life.

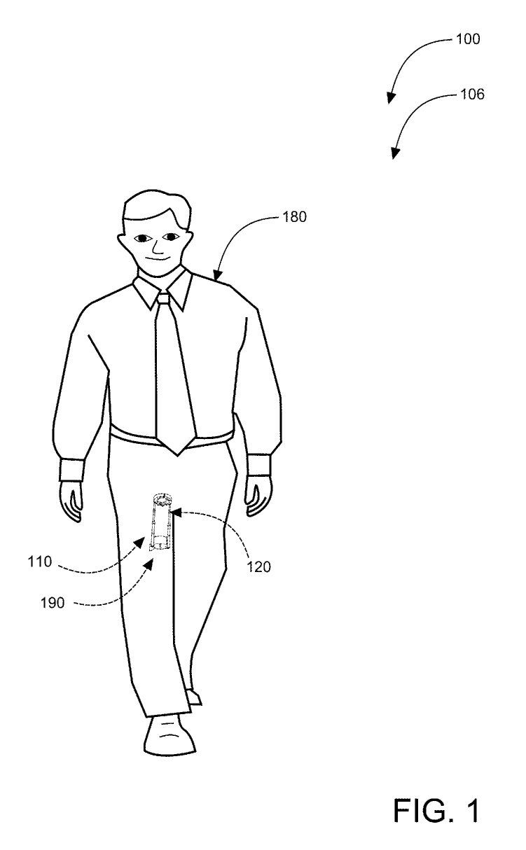

Referring now to the drawings by numerals of reference there is shown in FIG. 1, a perspective view illustrating penis stretching system 100 in ‘in-use’ condition 106 according to an embodiment of the present invention.

Penis stretching system 100 provides for safe, incremental stretching of penis 190 to increase a girth and length of penis 190. The incremental stretching may be provided as Eukaryotic cells of penis 190 are torn and repaired. Penis stretching system 100 preferably comprises: penis stretcher assembly 110 including; cylindrically-shaped frame 120; adjustable securing strap 160; and at least one vibrator 170. Cylindrically-shaped frame 120 preferably includes proximate end 122 comprising a base engager 130; distal end 140 comprising shaft engager 142; telescoping legs 150. Vibrator 170 preferably provides vibration to provide comfort (and/or stimulation means) for user-wearer 180. Penis stretcher assembly 110 comprises cylindrically-shaped frame 120, adjustable securing strap 160 and vibrator 170 in combination.

Proximate end 122 of cylindrically-shaped frame 120 preferably comprises first half 124 and second half 126 of base engager 130 such that proximate end 122 is adjustable to fit about base 192 of penis 190 adjacent scrotum 194. Distal end 140 of cylindrically-shaped frame 120 preferably comprises arc 144 comprising right half-arc 146 and left half-arc 148. Right half-arc 146 and left half-arc 148 are adjustable in relation to each other providing that distal end 140 is able to friction engage shaft 196 of penis 190 adjacent and under knob 198. Proximate end 122 and distal end 140 may be connected via telescoping legs 150. Telescoping legs 150 are able to provide extension and retraction of penis stretcher assembly 110. Adjustable securing strap 160 and distal end 140 engage shaft 196 of penis 190. Adjustable securing strap 160 is held stationary by left half-arc 148 and is insertable through right half-arc 146 to create a contact-fit about shaft 196 of penis 190.

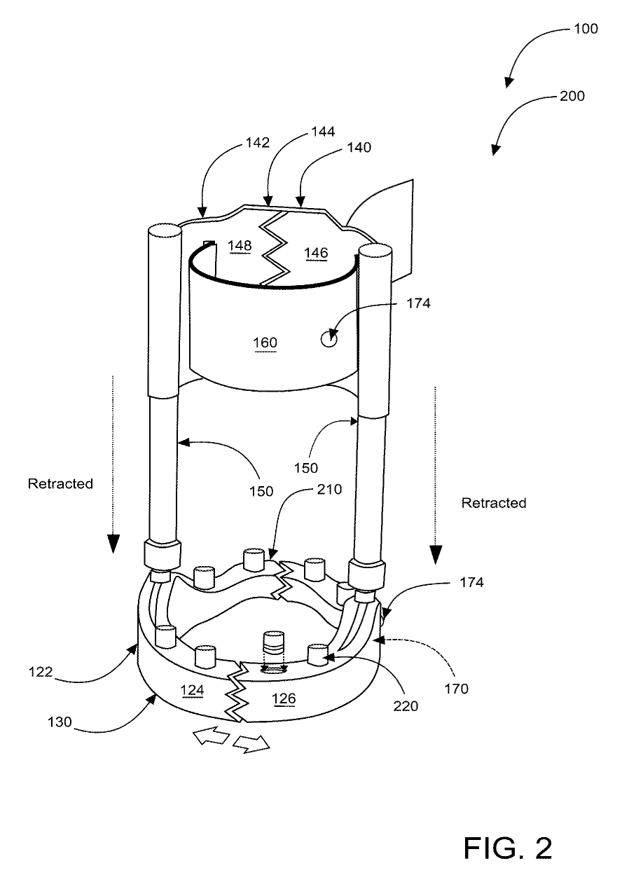

Referring now to FIG. 2, a perspective view illustrating penis stretcher assembly 110 of penis stretching system 100 in retracted condition 200 according to an embodiment of the present invention of FIG. 1.

Vibrator 170 preferably permits comfort-controlled incremental stretching of penis 190 and promotes blood flow and may act in the capacity of a stimulator to encourage penis 190 to reach a hardened condition. Vibrator 170 also preferably comprises activator button 174. Activator button 174 for vibrator 170 may be adjustable.

Proximate end 122 of the present invention is extendable and retractable in diameter via first half 124 and second half 126 of base engager 130 such that proximate end 122 is width adjustable to fit about base 192 of penis 190 adjacent scrotum 194. Proximate end 122 comprises cammed upper surface 210 in preferred embodiments.

Penis stretcher assembly 110 further comprises removable studs 220; removable studs 220 able to be coupled to receiver inserts 214 located in cammed upper surface 210 in proximate end 122. Removable studs 220 preferably comprise plastic. Upon reading this specification, it should be appreciated that, under appropriate circumstances, considering such issues as user preferences, design preference, structural requirements, marketing preferences, cost, available materials, technological advances, etc., other material arrangements such as, for example, rubber, vinyl, etc., may be sufficient.

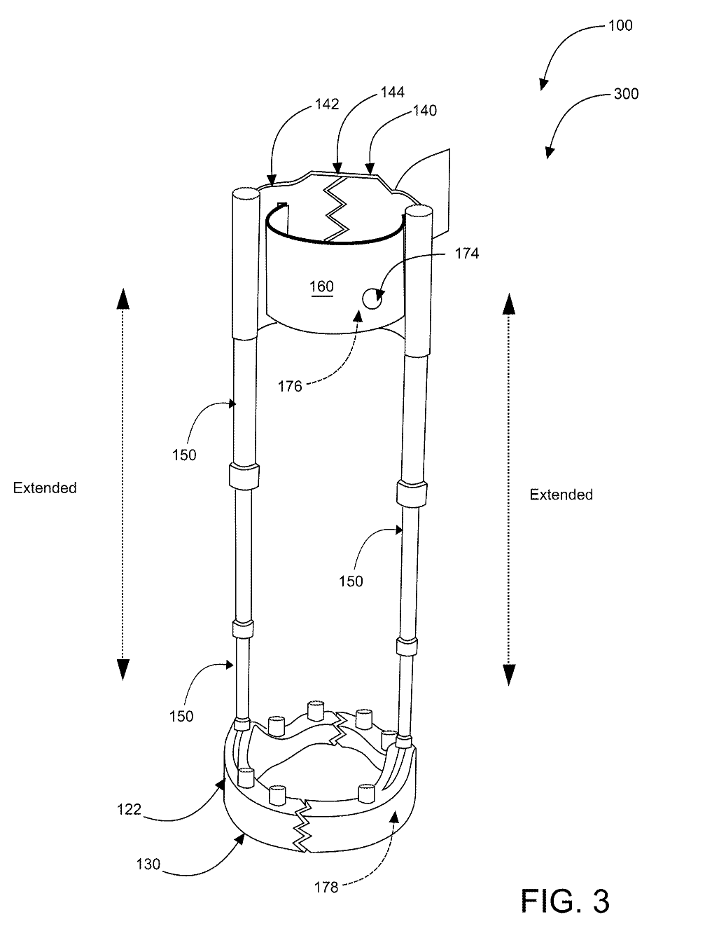

Referring now to FIG. 3, a perspective view illustrating penis stretcher assembly 110 of penis stretching system 100 in extended condition 300 according to an embodiment of the present invention of FIG. 1.

Telescoping legs 150 may be lockable and telescoping legs 150 preferably comprise rigid rubber material such that the present invention is comfortable and safe in-use. Upon reading this specification, it should be appreciated that, under appropriate circumstances, considering such issues as user preferences, design preference, structural requirements, marketing preferences, cost, available materials, technological advances, etc., other material arrangements such as, for example, plastic, metal, non-metal, composites, etc., may be sufficient.

Adjustable securing strap 160 is able to accommodate a circumcised and/or an uncircumcised penis 190 making it practical for use by virtually all males. Adjustable securing strap 160 preferably comprises stretchy (soft) rubber. Upon reading this specification, it should be appreciated that, under appropriate circumstances, considering such issues as user preferences, design preference, structural requirements, marketing preferences, cost, available materials, technological advances, etc., other material arrangements such as, for example, elastic material, plastic, etc., may be sufficient Additionally, penis stretcher assembly 110 may also useful to straighten curves out of penis 190, the curves in penis 190 caused by Peyronie's Disease. Peyronie's Disease is a connective tissue disorder involving the growth of fibrous plaques in the soft tissue of penis 190 affecting up to 10% of men. Specifically, scar tissue forms in the tunica albuginea, the thick sheath of tissue surrounding the corpora cavernosa causing pain, abnormal curvature, erectile dysfunction, indentation, loss of girth and shortening of penis 190. Although the popular conception of Peyronie's Disease is that it always involves curvature of penis 190, the scar tissue sometimes causes divots or indentations rather than curvature. The condition may also make sexual intercourse painful and/or difficult, though many men report satisfactory intercourse in spite of the disorder. Although it can affect men of any race and age, it is most commonly seen in Caucasian males above the age of 40, especially those of blood type A+, but has been seen in men as young as 18. In this way the present invention serves to promote penis 190 health in affected users.

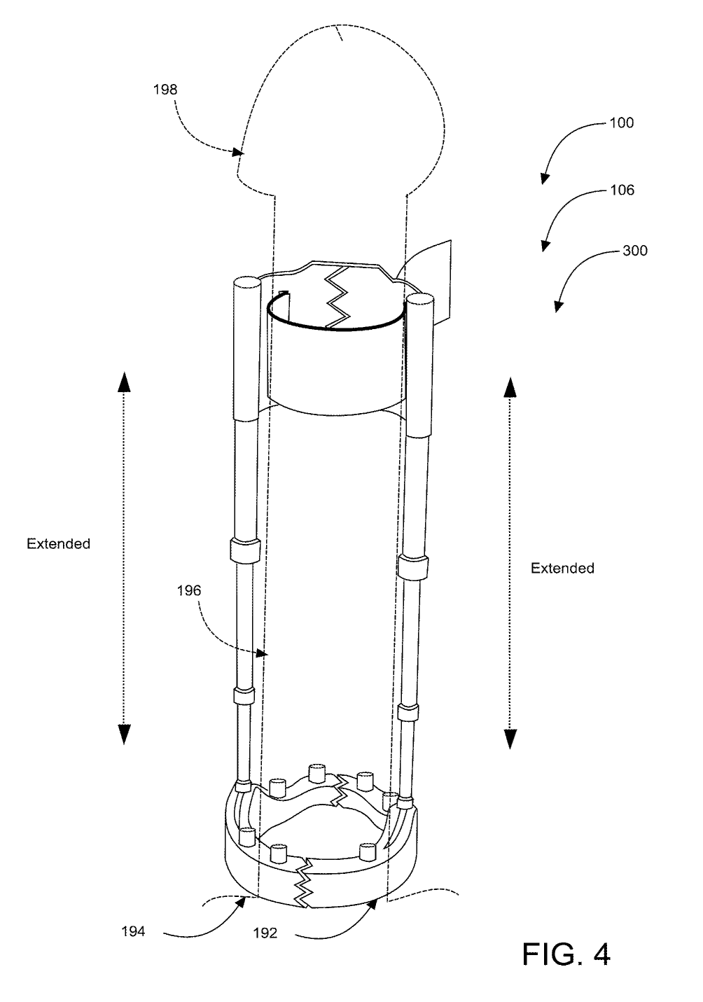

Referring now to FIG. 4, a perspective view illustrating penis stretcher assembly 110 of penis stretching system 100 in extended condition 300 about penis 190 according to an embodiment of the present invention of FIGS. 1 and 3.

Penis stretcher assembly 110 can be worn while user-wearer 180 is in a non-standing position or in a standing position as also shown in FIG. 1. Penis stretcher assembly 110 can be worn while user-wearer 180 is in a standing position and a non-standing position through a time duration during the course of an entire day because of the comfortable materials used. In this way the extended duration promotes quicker and more reliable stretching of penis 190. Adjustable securing strap 160 may also comprise triple-padded protection since adjustable securing strap 160 may be strapped to such sensitive area(s) on penis 190 of user-wearer 180.

Proximate end 122 and distal end 140 each may comprise vibrator(s) 170 comprising first vibrator 176 and second vibrator 178, first vibrator 176 and second vibrator(s) 178 being independently activatable. The present invention may use only one vibrator 170 in certain embodiments and other embodiments may comprise no vibration means.

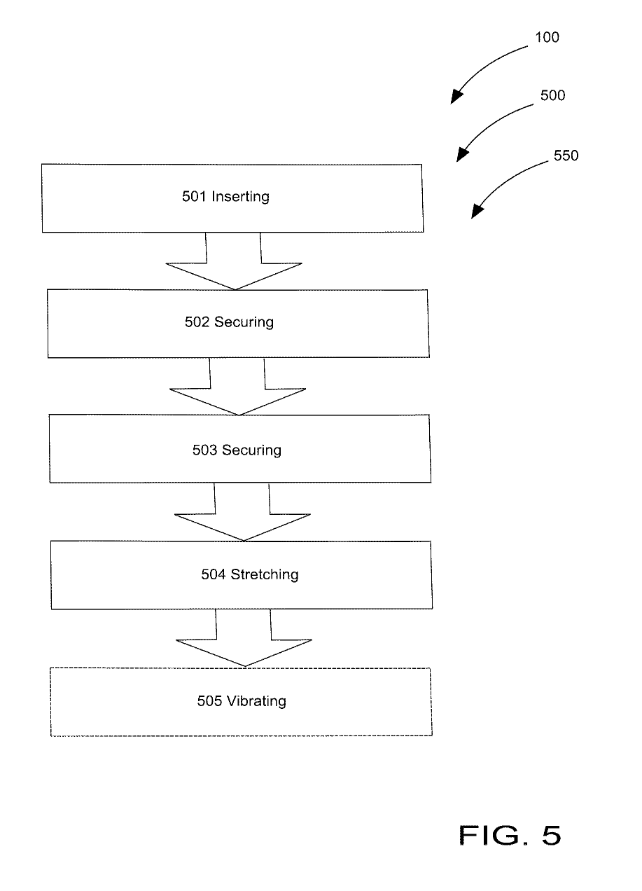

Referring now to FIG. 5, a flowchart 550 illustrating a method of use 500 for penis stretcher assembly 110 of penis stretching system 100 according to an embodiment of the present invention of FIGS. 1-4.

A method of using (enabling method of use 500) penis stretcher assembly 110 preferably comprises the steps of: step one 501 inserting penis 190 into penis stretcher assembly 110; step two 502 securing adjustable securing strap 160 and distal end 140 to shaft 196 of penis 190; step three 503 securing proximate end 122 to base 192 of penis 190; step four 504 stretching penis 190 using telescoping legs 150 throughout a time period; and step five 505 vibrating proximate end 122 and distal end 140 while stretching penis 190 to increase blood flow to accelerate repair of cells torn during the stretching step. Vibration step may comprise a stimulation step.

It should be noted that step 505 is an optional step and may not be implemented in all cases. Optional steps of method 500 are illustrated using dotted lines in FIG. 5 so as to distinguish them from the other steps of method 500.

It should be noted that the steps described in the method of use can be carried out in many different orders according to user preference. The use of “step of” should not be interpreted as “step for”, in the claims herein and is not intended to invoke the provisions of 35U.S.C. §112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A penis stretching system comprising:
a penis stretcher assembly including;
a cylindrically-shaped frame having;
a proximate end comprising a base engager;
a distal end comprising a shaft engager;
telescoping legs;
an adjustable securing strap; and
at least one vibrator;
wherein said penis stretcher assembly comprises said cylindrically-shaped frame, said adjustable securing strap and said vibrator in combination;
wherein said proximate end comprises a first half and a second half suitable as said base engager such that said proximate end is adjustable to fit about a penis base adjacent a scrotum;
wherein said distal end comprises an arc comprising a right half-arc and a left half-arc;
wherein said right half-arc and said left half-arc are adjustable in relation to each other providing that said distal end is able to friction engage a penis shaft adjacent and under a knob;
wherein said adjustable securing strap and said distal end engage said penis shaft;
wherein said adjustable securing strap is held stationary by said left half-arc and is insertable through said right half-arc to create a contact-fit about said penis shaft;
wherein said proximate end and said distal end are connected via said telescoping legs;
wherein said telescoping legs are able to provide extension and retraction of said penis stretcher assembly;
wherein said vibrator provides vibration to provide comfort and stimulation for a user-wearer; and
wherein said penis stretching system provides for safe, incremental stretching of a penis to increase a girth and length of said penis.

2. The penis stretching system of claim 1 wherein said incremental stretching is provided as Eukaryotic cells of said penis are torn and repaired.

3. The penis stretching system of claim 1 wherein said telescoping legs are lockable.

4. The penis stretching system of claim 1 wherein said penis stretcher assembly is useful to straighten curves out of said penis, said curves in said penis caused by Peyronie's Disease.

5. The penis stretching system of claim 2 wherein said vibrator permits comfort-controlled said incremental stretching of said penis and promotes blood flow.

6. The penis stretching system of claim 5 wherein said vibrator comprises an activator button.

7. The penis stretching system of claim 6 wherein said activator button for said vibrator is adjustable.

8. The penis stretching system of claim 1 wherein said proximate end comprises a cammed upper surface.

9. The penis stretching system of claim 8 wherein said penis stretcher assembly further comprises removable studs, said removable studs able to be coupled to receiver inserts located in said cammed upper surface in said proximate end.

10. The penis stretching system of claim 1 wherein said proximate end is extendable and retractable in diameter via said first half and said second half of said base engager such that said proximate end is width adjustable to fit about said penis base adjacent said scrotum.

11. The penis stretching system of claim 1 wherein said telescoping legs comprise rigid rubber material.

12. The penis stretching system of claim 1 wherein said adjustable securing strap is able to accommodate a circumcised and an uncircumcised said penis.

13. The penis stretching system of claim 12 wherein said adjustable securing strap comprises rubber.

14. The penis stretching system of claim 1 wherein said penis stretcher assembly can be worn while said user-wearer is in a non-standing position.

15. The penis stretching system of claim 1 wherein said penis stretcher assembly can be worn while said user-wearer is in a standing position.

16. The penis stretching system of claim 1 wherein said penis stretcher assembly can be worn while said user-wearer is in a standing position and a non-standing position through a time duration.

17. The penis stretching system of claim 1 wherein said proximate end and said distal end each comprise said vibrators comprising a first vibrator and a second vibrator, said first vibrator and said second vibrator(s) being independently activatable.

18. A penis stretching system comprising:
a penis stretcher assembly including;
a cylindrically-shaped frame having;
a proximate end comprising a base engager;
a distal end comprising a shaft engager;
telescoping legs;
an adjustable securing strap; and
two vibrators;
wherein said penis stretcher assembly comprises said cylindrically-shaped frame, said adjustable securing strap and said vibrators in combination;
wherein said proximate end comprises a first half and a second half suitable as said base engager such that said proximate end is adjustable to fit about a penis base adjacent a scrotum;
wherein said proximate end is extendable and retractable in diameter via said first half and said second half of said base engager such that said proximate end is width adjustable to fit about said penis base adjacent said scrotum;
wherein said proximate end comprises a cammed upper surface;
wherein said penis stretcher assembly further comprises removable studs, said removable studs able to be coupled to receiver inserts located in said cammed upper surface in said proximate end;
wherein said cammed upper surface in said proximate end further comprises guide tracks allowing said telescoping legs to move along said cammed upper surface;
wherein said distal end comprises an arc comprising a right half-arc and a left half-arc;
wherein said right half-arc and said left half-arc are adjustable in relation to each other providing that said distal end is able to friction engage a penis shaft adjacent and under a knob of a penis;
wherein said adjustable securing strap and said distal end in combination engage said penis shaft;
wherein said adjustable securing strap is held stationary by said left half-arc and is insertable through said right half-arc to create a contact-fit of said adjustable securing strap about said penis shaft;
wherein said adjustable securing strap is able to accommodate a circumcised said penis, and an uncircumcised said penis;
wherein said adjustable securing strap comprises rubber;
wherein said penis stretcher assembly comprises triple-padded protection;
wherein said proximate end and said distal end are connected via said telescoping legs;
wherein said telescoping legs are able to provide extension and retraction of said penis stretcher assembly;
wherein said telescoping legs are lockable via locking detents at a plurality of positions;
wherein said telescoping legs comprise rigid rubber material;
wherein said vibrators provide vibration to provide comfort and stimulation for a user-wearer; wherein incremental stretching is provided as Eukaryotic cells of said penis are torn and repaired over an extended time period;
wherein said vibrators permit comfort-controlled said incremental stretching of said penis and promotes blood flow;
wherein said proximate end and said distal end each comprise said vibrators comprising a first vibrator and a second vibrator, respectively, said first vibrator and said second vibrator(s) being independently activatable;
wherein said first vibrator and said second vibrator(s) each comprise activator buttons;
wherein said activator button for said first vibrator and said second vibrator(s) are adjustable to adjust a frequency of said vibration;
wherein said penis stretching system provides for safe, said incremental stretching of said penis to increase a girth and length of said penis; and
wherein said penis stretcher assembly can be worn while said user-wearer is in a standing position and a non-standing position throughout a time duration.

19. A method of using a penis stretcher assembly comprising the steps of:
inserting a penis into a penis stretcher assembly;
securing an adjustable securing strap and a distal end to a shaft of said penis;
securing a proximate end to a base of said penis; and
stretching said penis using telescoping legs throughout a time period.

20. The method of claim 19 further comprising the step of vibrating said proximate end and said distal end while stretching said penis to increase blood flow to accelerate repair of cells torn during said stretching step.
